(19) United States
(12) Patent Application Publication
(10) Pub. No.: US 2022/0062440 A1
(43) Pub. Date: Mar. 3, 2022

(54) OLIGOMER-SELECTIVE FLUORESCENT INDICATOR DYES

(71) Applicant: University of South Florida, Tampa, FL (US)

(72) Inventor: Martin Matthias Muschol, Tampa, FL (US)

(21) Appl. No.: 17/312,690

(22) PCT Filed: Dec. 13, 2019
(86) PCT No.: PCT/US2019/066355

§ 371 Date: Jun. 10, 2021

Related U.S. Application Data
(60) Provisional application No. 62/779,676, filed on Dec. 14, 2018.

(51) Int. Cl. A61K 49/00; C09B 11/08; C09B 11/12; G01N 33/68

(52) U.S. Cl. CPC A61K 49/0023; C09B 11/08; G01N 2333/4709; G01N 33/6839; C09B 11/12

(57) ABSTRACT

Amyloids have been known to arise from many different proteins and polypeptides. These polypeptide chains generally form β-sheet structures that aggregate into long fibers; however, identical polypeptides can fold into multiple distinct amyloid conformations. The diversity of the conformations may have led to different forms of the prion diseases. In particular, large populations of small globular amyloid oligomers (gOs) and curvilinear fibrils (CFs) precede the formation of late-stage rigid fibrils (RFs), and have been implicated in amyloid toxicity. As disclosed herein, triarylmethane f dye crystal violet is a highly selective indicator of gOs and CFs. Therefore, disclosed herein are compositions, kits, and methods for detecting amyloids in a tissue, either in vitro or in vivo.

### CROSS-REFERENCE TO RELATED APPLICATIONS

This application claims benefit of U.S. Provisional Application No. 62/779,676, filed Dec. 14, 2018, which is hereby incorporated herein by reference in its entirety.

### STATEMENT REGARDING FEDERALLY SPONSORED RESEARCH OR DEVELOPMENT

This invention was made with Government Support under Grant No. GM097723 awarded by the National Institutes of Health. The Government has certain rights in the invention.

### BACKGROUND

Amyloidosis is a group of diseases caused by the formation of amyloid aggregates [Chiti, F., et al. Annu. Rev. Biochem. (2006) 75: 333-366]. These amyloid aggregates are believed to fall into two main categories: globular oligomers and curvilinear fibrils (gO/CFs) or “traditional” rigid fibrils (RFs) [Gosal, W. S., et al. J. Mol. Biol. (2005) 351: 850-864. T. Miti et al. Biomacromolecules (2015) 16: 326-335]. A major focus of research is to unravel the mechanisms and kinetics of formation for these distinct amyloid aggregate species. The most commonly used technique for monitoring amyloid assembly kinetics is the amyloid indicator dye Thioflavin-T (ThT). A significant drawback, however, is that ThT fluorescence responds to both gO/CF and RF aggregates, albeit with different sensitivities [Joseph Foley, et al. J. Chem. Phys. (2013) 139:121901]. This makes the decomposition of ThT kinetics into its gO/CF and RF components difficult to impossible. Yet, even screening protocols for identifying the selectivity of dyes for one over the other amyloid species are lacking. There is an unmet need for a method to selectively determine the presence of gO/CF species in samples.

### SUMMARY

Amyloids have been known to arise from many different proteins and polypeptides. These polypeptide chains generally form β-sheet structures that aggregate into long fibers; however, identical polypeptides can fold into multiple distinct amyloid conformations. The diversity of the conformations may have led to different forms of the prion diseases. In particular, large populations of small globular amyloid oligomers (gOs) and curvilinear fibrils (CFs) are often found to precede the formation of late-stage rigid fibrils (RFs), and have been implicated as major contributors to amyloid toxicity.

As disclosed herein, the triarylmethane dye crystal violet (also known as tris(4-(dimethylamino)phenyl)methylium chloride, methyl violet 10B, or hexamethyl pararosaniline chloride) is a highly selective indicator of gOs and CFs. Therefore, disclosed herein are compositions, kits, and methods for selectively detecting amyloids in a tissue, either in vitro or in vivo.

For example, disclosed herein is a composition that comprises a gO/CF-selective dye.

Also disclosed is a kit for detecting amyloids in a tissue that contains a gO/CF-selective dye.

Optionally, in either of the two embodiments above, the composition and/or kit may further comprise an RF-selective dye.

Therefore, disclosed herein are methods to diagnose early stages of neurological disease involving amyloid B oligomers, such as Alzheimer's, Lewy Body dementia, etc., that involve detecting gO/CF in the subject or in a sample from the subject.

For example, disclosed is an in vitro method for detecting amyloids in a biological sample, such as a tissue or bodily fluid sample that involves contacting the tissue with a gO/CF-selective dye and assaying the sample for presence of the gO/CF-selective dye. In some embodiments, the method further involves contacting the tissue with an RF-selective dye and assaying the sample for presence of the RF-binding dye.

Also disclosed is an in vivo method for detecting amyloids in a subject that involves administering to the subject a composition comprising a gO/CF-selective dye and assaying the subject for presence of the gO/CF-selective dye. In some embodiments, the method further involves administering to the subject a composition comprising an RF-selective dye and assaying the subject for presence of the RF-binding dye. Therefore, in some embodiments, the gO/CF-selective dye and/or the RF-selective dye is tethered to a positron-emission tomography (PET) or magnetic resonance imaging (MRI) probe.

In some embodiments of the above compositions and methods, the RF-selective dye can be thioflavin T. In some embodiments of the above compositions and methods, the gO/CF-selective dye comprises a triarylmethane dye, such as a methyl violet dye, such as crystal violet. In one embodiment, the triarylmethane dye is crystal violet. Additional RF-selective and gO/CF-selective dyes are described herein.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a semi-log plot of biphasic growth kinetics for 350 μM HEWL (pH 2, 400 mM NaCl, 52° C.) monitored with ThT and a globular oligomer indicator (gOI) crystal violet. ThT shows the expected biphasic behavior as the sample grows gO/CFs up to a first plateau, with a second upswing and plateau indicating the onset of RF nucleation and growth. In contrast, the globular oligomer indicator crystal violet responds nearly exclusively to the initial (oligomer) phase with little or no response to the onset of RF formation. FIGS. 1B-1E are AFM images taken at 4 hours (FIG. 1B), 21 hours (FIG. 1C), 45 hours (FIG. 1D) and 92 hours (FIG. 1E) indicating the presence of gO/CFs (FIGS. 1B-D) and emergence of RFs (FIG. 1E).

FIGS. 2A to 2C are atomic force microscope (AFM) images of rigid fibrils (RFs, FIG. 2A), globular oligomers (gOs, FIG. 2B), and curvilinear fibrils (CFs, FIG. 2C). Scale bar=250 nm.

FIG. 3A shows transition from sigmoidal to biphasic kinetics observed with ThT for HEWL amyloid formation (pH 2, 52° C.). FIG. 3B shows corresponding kinetics recorded with the disclosed globular oligomer indicator crystal violet under identical conditions.

FIGS. 4A and 4B show correlation between ThT and crystal violet signals.

FIGS. 5A and 5B show a schematic of kinetic transition in amyloid assembly used to identify oligomerselective dyes (OSDs). FIG. 5A shows low protein concentrations amyloid fibril growth, as monitored with thioflavin T (ThT), displays the ubiquitous sigmoidal growth kinetics of standard amyloid fi-brils (referred to as rigid fibrils or RFs). There is discernible presence of long-lived globular oli-gomers (gOs). FIG. 5B shows crossing a well-defined threshold protein concentration, amyloid formation proceeds via biphasic growth. The lag phase is now replaced by a progressively more prominent initial growth phase that can reach its own primary plateau. This initial phase in-dicates formation of significant populations of globular oligomers (gOs) which tend to polymerize into curvilinear fibrils (CFs). The second ThT upswing, in turn, indicates the nucleation and growth of rigid fibrils (RFs) against a background of residual gOs and CFs. Monitoring dye re-sponses in these two distinct amyloid assembly regimes can identify oligomer-selective dyes (OSDs). OSD responses (shown here as dash-dot lines) to RF formation in both the sigmoidal and biphasic regime will be much reduces, while they'll have a significant response to the initial, oligomer-dominated phase during biphasic growth.

FIGS. 6A to 6E show transition from fibril-dominated sigmoidal to oligomer-rich biphasic amyloid assembly kinetics in hen egg-white lysozyme and dimeric Aβ40. The formation of globular oligomers (gOs) and their curvilinear fibrils (CFs) induces a sharp transition in amyloid assembly kinetics in (FIGS. 6A-6C) hen egg-white lysozyme (hewL) and (FIGS. 6D, 6E) a dimeric Aβ40 construct (dimAβ). Below a threshold monomer concentration, amyloid assem-bly monitored with ThT displays well-defined sigmoidal kinetics. This reflects the nucleated polymerization of rigid fibrils (RFs). In this regime atomic force microscopy (AFM) only detects monomers (IB) in the lag phase and fibrils thereafter (II_{B}). Above the threshold concentration, ThT kinetics turns biphasic, with the initial phase yielding significant populations of gO/CFs (I_{C}, II_{C} or I_{E}-IV_{E}). Rigid fibrils (RFs) now only emerge during the second ThT upswing (III_{C}, IV_{C} or V_{E}). The absence or presence of the weak oligomer response by ThT is underscored by using a semi-log scale in these plots.

FIGS. 7A to 7D show multiple fluorescent dyes show (weak) select responses to oligomer-dominated phase of biphasic amyloid assembly by hewL. Kinetics responses of thioflavin T (FIG. 7A), the amyloid dye X-34 (FIG. 8B, λₑₓ=370 nm, λₑₘ=482 nm), acridine orange (FIG. 8C, λₑₓ=495 nm, λₑₘ=532 nm), and acid fuchsin (FIG. 8D, λₑₓ=560 nm, λₑₘ=590 nm) to amyloid growth of hewL at the indicated concentrations (in mg/ml) and under fixed solution conditions (pH 2, 52° C., 450 mM NaCl). Traces (0.3 & 0.6 mg/ml vs. blue traces 2 & 4 mg/ml) represent sigmoidal vs. biphasic growth conditions. Black traces are dye controls recorded from buffer solution. For overall comparison, ThT traces are displayed on a linear scale, which obscures its weak biphasic response. Dye concentrations were 15 μM. Sharp initial transients result from the temperature dependence of dye fluorescence upon heating to 52° C.

FIGS. 8A and 8B show Crystal Violet as oligomer-selective dye. FIG. 8A shows fractional change of thioflavin T (15 μM, open symbols) vs. crystal violet (5 μM, filled symbols) fluorescence during sigmoidal vs. biphasic amyloid assembly (5 mg/ml hewL, pH 2, 52° C., in the presence of either 150 or 450 mM NaCl). FIG. 8B contains AFM images from aliquots taken during biphasic amyloid growth in FIG. 8A from the CV wells at the indicated time points. As can be seen, CV did not alter the types of aggregates observed in the two phases of biphasic amyloid assembly under comparable conditions (see FIG. 6C)

FIGS. 9A to 9E show a switch from sigmoidal to biphasic amyloid growth of Aβ42 & Aβ40. FIG. 9A shows transition in Aβ42 amyloid growth at pH 7.4, no salt from sigmoidal to bi-phasic kinetics. FIGS. 9B and 9C shows transition in Aβ40 amyloid growth at pH 7.4 at either no salt (FIG. 9B) or 150 mM NaCl (FIG. 9C) (data in FIG. 9C show ThT fractional change). Notice the significant increase in gO/CF amplitude from FIG. 9B to 9C. FIGS. 9D and 9E contain TEM images from aliquots of samples in FIG. 9B. FIGS. 9D and 9E show RFs following sigmoidal growth at 50 μM Aβ40 (FIG. 9D) vs. gO/CFs at 90 μM Aβ40 (FIG. 9E), transforming into mixtures of gO/CF and RFs after 6 days. Note: dense uranyl acetate staining and fractal-like geometries are common features of gO/CF in TEM.

FIGS. 10A and 10B show correlation of Aβ40 kinetics and aggregate morphologies. FIG. 10A shows sigmoidal and biphasic kinetics of Aβ40 grown at 10 vs. 85 μM under near physiological solution conditions (pH 7.4, 150 mM NaCl, 27° C.). FIG. 10B shows corresponding aggregates morphologies of aliquots removed at the indicated time points and imaged with AFM. As with hewL and dimAβ, gO/CF aggregates persist long into the RF nucleation and growth phase. Images are 5 μm on a side

FIGS. 11A and 11B show Aβ purification for Dye Screening Assay. FPLC elution profiles of Aβ40 (FIG. 10A) and (B) Aβ42 (FIG. 11B) with monomers eluting at 14 mL. Both peptides were dissolved in 100 mM NaOH and the size exclusion column was equilibrated with 35 mM Na₂HPO₄ buffer at pH 11. The monomer fraction for either peptide is collected for kinetic experiments.

FIG. 12 shows an example layout of an assay plate for dye screening. The response of a given dye is monitored at four different Aβ40 concentrations, two under biphasic (80 & 120 μM) and two under sigmoidal (10 & 20 μM) at pH 7.4 and 150 mM NaCl (see FIG. 9C). This allows for measurements with 3 different dyes and the corresponding ThT control per 96-well assay plate

FIGS. 13A and 13B show detailed analysis of potential OSDs. FIG. 13A shows the effect of increasing concentrations of CV on ThT monitored sigmoidal growth by hewl. FIG. 13B shows temporal correlation of CV vs ThT signal in sigmoidal vs. biphasic growth regime. Measured CV and ThT responses.

FIGS. 14A to 14F show separation of dual-dye recordings into gO/CF and RF components. FIGS. 14A and 14B show HewL amyloid formation (pH 2, 450 mM NaCl, T=52° C.) at concentrations below (orange) and above (blue) the COC, simultaneously monitored with ThT (FIG. 14A, 15 μM) and CV (FIG. 14A, 5 μM). Traces are the average of three recordings from separate wells. FIGS. 14C and 14D show the correlation of the fractional changes of ThT vs. CV responses during sigmoidal (FIG. 14C) vs. biphasic growth kinetics (FIG. 14D). The r-coefficient indicates the ratio of the CV vs ThT response amplitude in the linear re-gime of each plot. FIGS. 14E and 14F show superposition of the CV (solid lines) and ThT (dashed lines) response, after matching the CV data using the r-factor determined in FIGS. 14C and 14D, respectively. FIG. 14F also shows subtracting the matched CV from the ThT trace in the biphasic regime recovers a perfectly sigmoidal trace consistent with the emergence of RFs. The CV trace reflects the corresponding growth and subsequent decay of gO/CFs.

FIGS. 15A to 15F show transition from sigmoidal to biphasic kinetics and associated gO/CF formation for Aβ40 and Aβ42. FIGS. 15A and 15B show transition in Aβ40 (FIG. 15A) and Aβ42 (FIG. 15B) growth kinetics from pure sigmoidal to biphasic kinetics (pH 7.4, no salt). Semilog plot emphasizes weak ThT response during gO/CF phase. FIG. 15C shows ThT fractional change during Aβ40 growth in physiological saline. Notice the significant increase in gO/CF amplitude relative to FIG. 15A. FIGS. 15D to 15F show TEM images of samples of Aβ40 RFs following sigmoidal growth at 50 μM (FIG. 15D) versus biphasic growth at 150 μM, with gO/CFs formed within 1.5 days (FIG. 15E), and mixtures of gO/CF and RFs after 6 days (FIG. 15F).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

Detection of Amyloid Oligomers

Amyloids are aggregates of proteins which can, under some circumstances, assume a conformation that allows many copies of that protein to form hydrogen bonds along their backbone and, thereby form fibrils. In the human body, amyloids have been linked to the development of various diseases. Pathogenic amyloids form when previously healthy proteins lose their normal physiological conformation and form fibrous deposits, either as extracellular plaques or intracellular inclusions (tangles, Lewis bodies) which can disrupt the healthy function of tissues and organs.

Such amyloids have been associated with more than 50 human diseases, known as amyloidoses, and play a critical role in multiple neurodegenerative disorders. Some amyloid proteins are infectious; these are called prions in which the infectious form can act as a template to convert other non-infectious proteins into infectious form. Amyloids may also have normal biological functions; for example, in the formation of fimbriae in some genera of bacteria, transmission of epigenetic traits in fungi, as well as pigment deposition and hormone release in humans.

Amyloids have been known to arise from many different proteins and polypeptides. These polypeptide chains generally form β-sheet structures that aggregate into long fibers; however, identical polypeptides can fold into multiple distinct amyloid conformations. The diversity of the conformations may have led to different forms of the prion diseases.

Frequently large populations of small globular amyloid oligomers (gOs) and curvilinear fibrils (CFs) precede the formation of late-stage rigid fibrils (RFs), and have been implicated as major contributors to amyloid toxicity. gOs and their associated CFs, often referred to as protofibrils, have been observed with large numbers of amyloid proteins and over a wide range of growth conditions. Substantial evidence suggests that early-stage gOs are potent sources of cytotoxicity in amyloid diseases. (U. Sengupta et al, EBioMedicine (2016) 6:42-49) Metastable oligomers also affect the aggregation of pharmaceuticals, and might hold answers to the question what distinguishes functional from pathological amyloid species. Formation of metastable precursors relates to a variety of physiochemical and biomedical problems. This includes metastable liquid phases as precursor of protein crystallization or sickle-cell hemoglobin fibrillation, as well as the significance of membrane-less organelles in promoting ALS fibril formation. Some amyloid oligomers themselves have been suggested to share characteristics of disordered liquid-like states.

In the clinical setting, amyloid aggregates are typically identified by a change in the fluorescence intensity of planar aromatic dyes such as thioflavin T, congo red, or NIAD-4. In general, this is attributed to the environmental change, as these dyes bind to the fibrillar beta-strands. However, these dyes preferentially respond to late stage RFs instead of gOs and CFs. Thus, they are not able to selectively determine the presence of gOs or CFs.

As disclosed herein, the triarylmethane dye crystal violet is a highly selective indicator of gOs and CFs. Therefore, disclosed herein are compositions, kits, and methods for detecting amyloids in a sample, either in vitro or in vivo, that involve a triarylmethane dye such as crystal violet, methyl violet, or a gO/CF-binding derivative thereof. The composition, kit, and/or method for detecting amyloids in a sample may further comprise a thioflavin T, congo red, or NIAD-4 dye, or an RF-binding derivative thereof. The methods involve assaying a sample which may contain bodily fluids or a tissue sample from a subject for detection of the aggregate-induced change in dye fluorescence.

In some embodiments, the in vitro method is performed on solutions, body fluids, or fixed or unfixed tissue sections, e.g. from live or cadaverous samples.

In some embodiments, the in vivo method is performed on subjects suspected of having amyloid deposits of some form.

Use of tissue sections allows for combined or separate staining. For example, in some embodiments, both dyes are used on the same tissue section and assayed simultaneously. In some cases, each dye is used separately on adjacent tissue sections. In some embodiments, the dyes are used serially on the same tissue section and assayed separately.

In some embodiments, the dye(s) are detected by light and/or fluorescent microscopy. For example, crystal violet dye fluorescence emitted at 630 nm is preferentially enhanced upon binding to gO/CFs when excited near 590 nm (or 560 nm); thioflavin T (ThT), upon binding to RFs, gives a strong fluorescence signal at approximately 482 nm when excited at 450 nm. In some embodiments, the dye(s) are detected by measuring fluorescence and/or absorbance according to standard methods known in the art.

gO/CF-Binding Dyes

Disclosed herein are triarylmethane dyes that preferentially respond to gO/CF amyloids. Triarylmethane dyes are synthetic organic compounds containing triphenylmethane backbones. Triarylmethane dyes can be grouped into families according to the nature of the substituents on the aryl groups. In some cases, the anions associated with the cationic dyes (e.g. crystal violet) vary even though the name of the dye does not. Often it is shown as chloride.

In some embodiments, the gO/CF-binding dye is a Methyl violet dye. Methyl violet dyes, such as Methyl violet 2B, Methyl violet 6B, and Methyl violet 10B, have dimethylamino groups at the p-positions of two aryl groups.

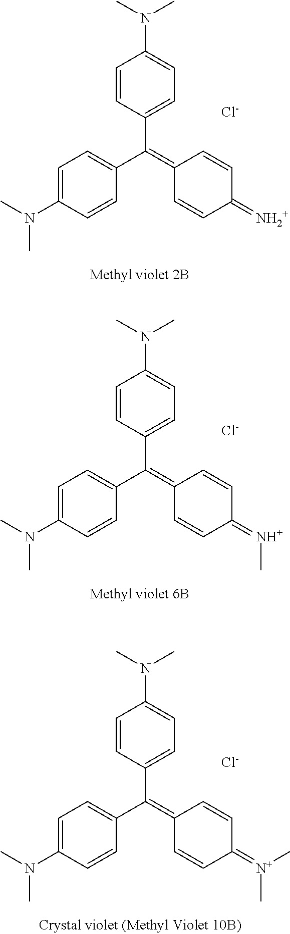

Crystal violet (also known as methyl violet 10B or hexamethyl pararosaniline chloride) is a triarylmethane dye predominantly used as a histological stain and in Gram's method of classifying bacteria.

In some embodiments, the disclosed gO/CF-binding dye is a compound selected from the group consisting of:

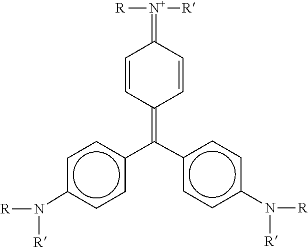

wherein each R and R′ is independently selected from the group consisting of hydrogen and C1-C6 linear or branched alkyl.

In some embodiments, the gO/CF-binding dye is a fuchsine dye, such as pararosaniline, fuchsine, new fuchsine, and fuchsine acid. In some embodiments, the gO/CF-binding dye is a phenol dye, such as phenolphthalein, phenol red, chlorophenol red, cresol red, bromocresol purple, and bromocresol green. In some embodiments, the gO/CF-binding dye is a malachite green dye, such as malachite green, Brilliant green (dye), and Brilliant Blue FCF. In some embodiments, the gO/CF-binding dye is a Victoria blue dye, such as Victoria Blue B, Victoria Blue FBR, Victoria blue BO, Victoria Blue FGA, Victoria blue 4 R, and Victoria blue R.

A number of possible routes can be used to prepare methyl violet dyes, such as crystal violet. The original procedure involved the reaction of dimethylaniline with phosgene to give 4,4′-bis(dimethylamino)benzophenone as an intermediate. This was then reacted with additional dimethylaniline in the presence of phosphorus oxychloride and hydrochloric acid.

The dye can also be prepared by the condensation of formaldehyde and dimethylaniline to give a leuco dye:

CH₂O+3C₆H₅N(CH₃)₂→CH(C₆H₄N(CH₃)₂)₃+H₂O

This colorless compound can then be oxidized to the colored cationic form: (A typical oxidizing agent is manganese dioxide).

CH(C₆H₄N(CH₃)₂)₃+HCl+1/2O₂→[C(C₆H₄N(CH₃)₂)₃]Cl+H₂O

RF-Binding Dyes

In some embodiments, the disclosed compositions, kits and methods can further comprise dyes that preferentially respond to RF amyloids, such as thioflavin T, congo red, NIAD-4 dye, or an RF-binding derivative thereof.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### A Selective Oligomer Dye as Companion for Thioflavin-T

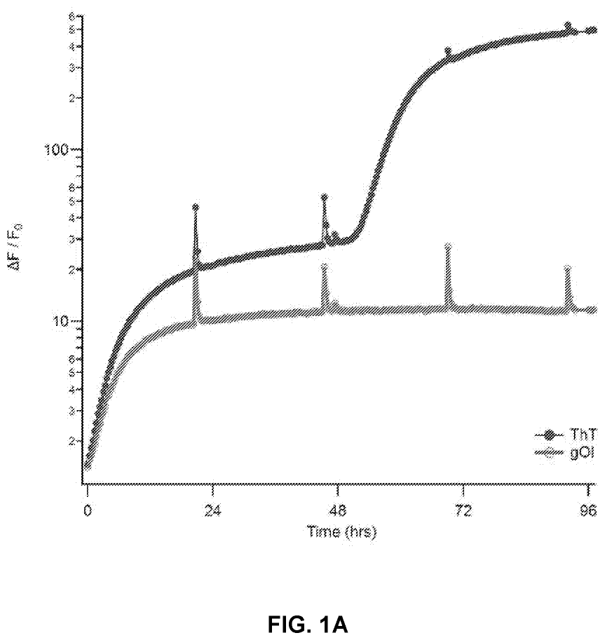

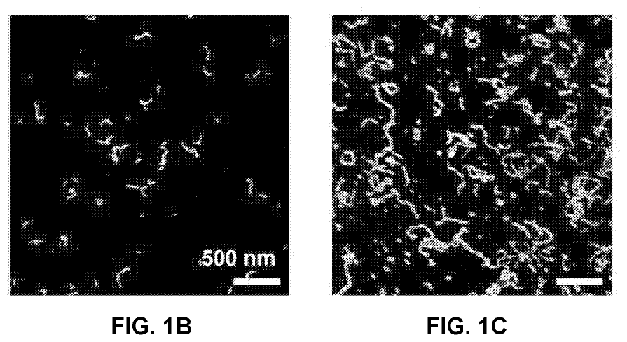

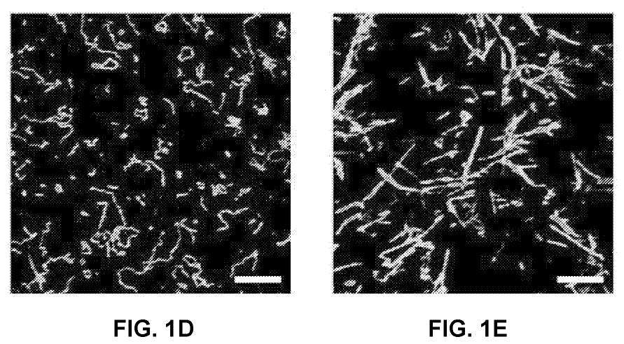

A kinetic assay that clearly delineates growth conditions dominated by gO/CF or RF formation as simple function of protein concentration was developed. This kinetic assay was used in conjunction with high-resolution (AFM or TEM) imaging of aggregate morphologies to identify dyes that display well-defined selectivity of gO/CFs over RFs (FIGS. 1A-1E).

The basis of the kinetic assay is the existence of a “critical oligomer concentration” or COC, distinct from and higher than the protein concentration required for fibril growth [Tatiana Miti, et al. Biomacromolecules (2015) 16:326-335]. Using both hen-egg white lysozyme and a dimeric Aβ construct, it was shown that crossing this COC causes a sharp transition in ThT kinetics from the ubiquitous “sigmoidal” growth kinetics of nucleated polymerization to lag-free bimodal kinetics. The initial phase of bimodal growth is dominated by gO/CF formation while the secondary upswing indicates RF nucleation and growth [Filip Hasecke, et al. Chem. Sci. (2018) 9: 5937-5948]. When below this COC HEWL grows RFS aggregates with a distinct lag phase before the start of growth. Monitoring dye responses both below and above the COC allows rapid determination whether a given fluorescent dye responds only to the initial gO/CF or subsequent RF phase of amyloid growth.

Using this kinetic assay with HEWL at pH 2 the transition in growth with both ThT (control) and several other molecular rotor dyes were monitored. Crystal violet was shown to closely match the kinetics of the initial gO/CF phase in ThT kinetics, but displays very weak responses upon the onset of RF formation. This “globular oligomer indicator” (GOI) would therefore complement the kinetic information available from ThT and enable to cleanly separate gO/CF from RF kinetics.

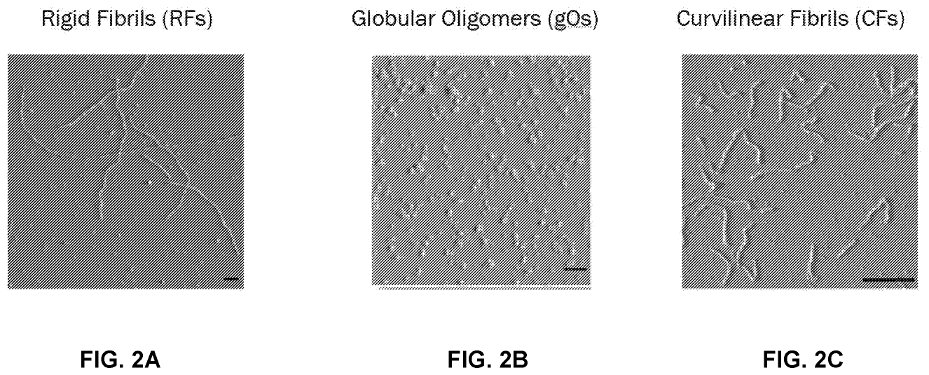

There are two main categories of amyloid aggregates: Long, Rigid Fibrils (RFs, FIG. 2A), Globular Oligomers (gOs, FIG. 2B), and Curvilinear Fibrils (CFs, FIG. 2C). Particularly gOs have been implicated as major contributors to amyloid diseases. However, there are few tools to monitor their growth behavior in isolation from RF growth.

Which of the species of aggregates is most disease-relevant is an ongoing question. The most common ways to confirm which species of amyloid aggregates have grown is with imaging (AFM, TEM), or with oligomer-specific antibodies (low time resolution). Being able to monitor which of these aggregates is forming during in vitro growth would be a powerful tool to understand the mechanisms underlying gO/CF vs. RF formation. Disclosed herein is a kinetic assay for screening dyes for their potential as selective indicators for either gO/CF or RF formation.

Kinetic transition is an indicator of oligomer formation (Hasecke F. et al., Chem. Sci., 2018 9:5937-5948). The onset of gO/CF formation is delineated by a threshold protein concentration referred to herein as the “critical oligomer concentration” or COC. Below the COC, RFs formation proceeds via nucleated polymerization and, therefore, displays sigmoidal growth kinetics with well-defined lag periods. No populations of gO/CFs are observed. In contrast, gO/CF formation above the COC has no lag periods and results in biphasic kinetics (saturating gO/CF kinetics is superimposed to sigmodal RF kinetics). AFM confirms that the initial phase represents gO/CF formation while the second phase indicates RF nucleation and growth. Using this transition from sigmoidal to biphasic kinetics provides an efficient assay for screening dyes for their response to these distinct aggregate species.

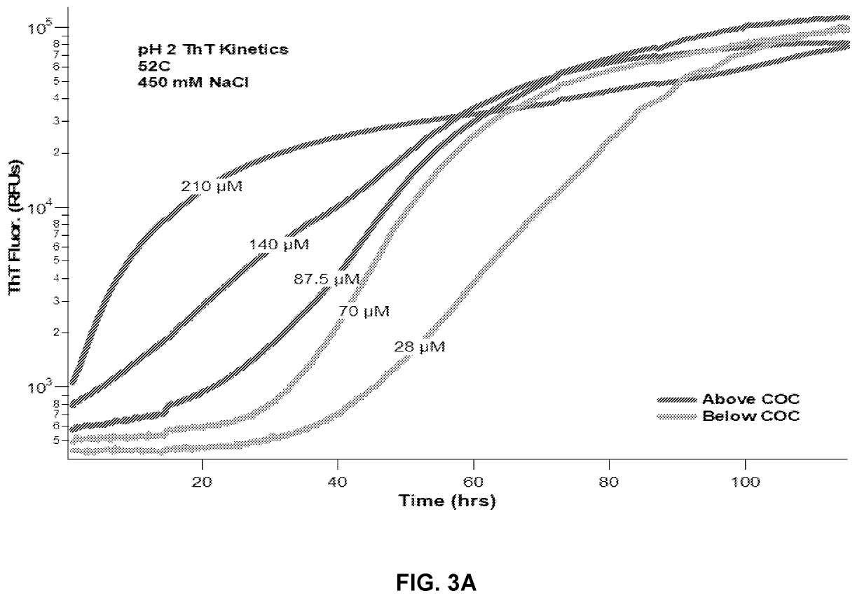

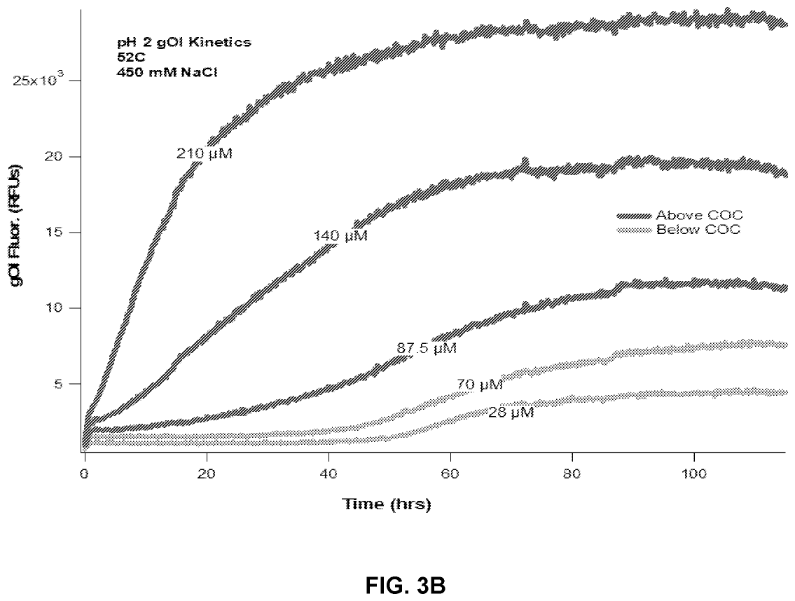

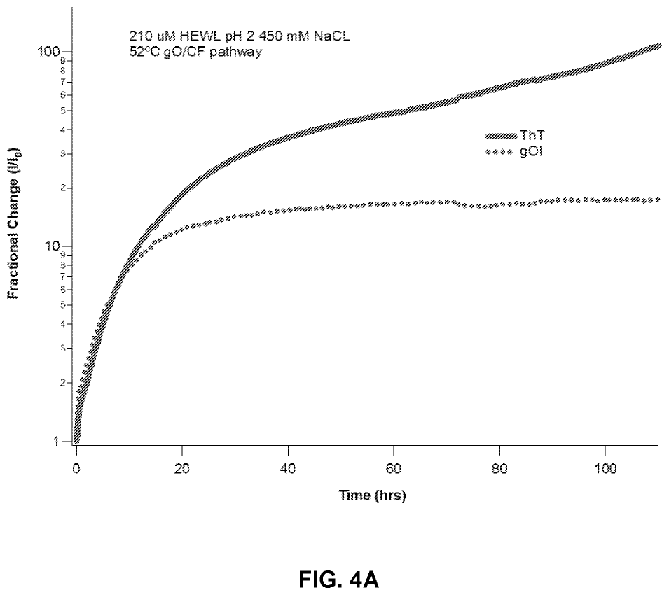

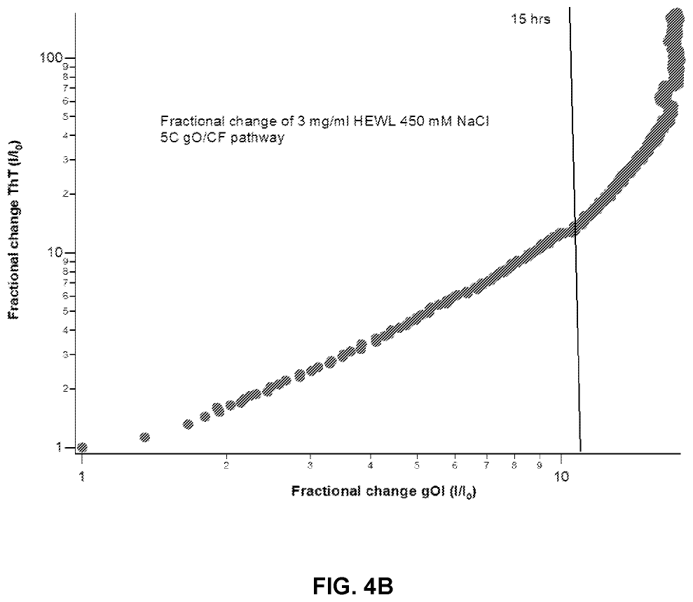

As disclosed herein, crystal violet is a globular oligomer indicator (GOI). As shown in FIGS. 3 and 4, ThT and GOI (crystal violet) have identical response to amyloid growth prior to the onset of RF formation. Thereafter crystal violet responses level off, while ThT continues to rise following RF nucleation.

### Example 2

Significance

Accumulating evidence implicates small globular oligomers both as cause of and biomarker for the clinical symptoms of Alzheimer's disease (AD) (Haass, C., et al. Nat Rev Mol Cell Biol., 2007. 8:101-112; Lesné, S., et al. Nature, 2006. 440:352-357; Tomic, J. L., et al. Neurobiology of Disease, 2009. 35:352-358; Dahlgren, K. N., et al. J Biol Chem., 2002. 277:36046-36053; Cleary, J. P., et al. Nature Neuroscience, 2005. 8:79-84; Pigino, G., et al. Proc Natl Acad Sci USA, 2009. 106:5907-5912; Kayed, R., et al. J Alzheimers Dis. 2013. 33:S67-S78; Serrano-Pozo, A., et al. Cold Spring Harb Perspect Med., 2011. 1:a006189). Efforts to translate this insight from basic research into clinical practice, however, suffer from a dearth of assays suitable for detecting and quantifying amyloid oligomers and their distributions ex vivo and, particularly, in vivo. Current techniques for detecting oligomers include atomic force microscopy (AFM) (Harper, J. D., et al. Chemistry and Biology, 1997. 4:119-125; Stine, W. B., Jr., et al. J Biol Chem., 2003. 278:11612-11622; Hill, S. E., et al. Biophysical Journal, 2009. 96:3781-3790), photo-induced cross-linking of unmodified proteins (PICUP) (Bitan, G., in Methods in Enzymology. 2006, Academic Press. p. 217-236), mass spectroscopy (Young, L. M., et al. J Am Chem Soc., 2014. 136:660-670), and most frequently immunostaining with conformation-dependent anti-oligomer antibodies (Kayed, R., et al. Molecular Neurodegeneration, 2007. 2:18; Kayed, R., et al., in Methods in Enzymology. 2006, Academic Press. p. 326-344). All of these methods suffer from limited temporal resolution, and only anti-oligomer antibodies are suitable for oligomer detection ex vivo (Schuster, J., et al., J Alzheimers Dis., 2016. 53:53-67). In addition, none of these approaches holds promise for detecting or imaging oligomers in vivo (e.g. via oligomer-selective PET tracers). This dearth of readily available and reliable probes of amyloid oligomers has prevented scientists from understanding the basic mechanisms underlying oligomer formation, from elucidating oligomer effects on cell viability, and from correlating disease symptoms with longitudinal studies of oligomer accumulation in vivo. These are all critical steps towards confirming the contributions of amyloid oligomers to AD and monitoring the efficacy of therapeutic interventions targeting AD oligomers.

One of the fundamental challenges in developing oligomer-selective assays is the inherent difficulty in isolating and stabilizing transient and metastable oligomer populations for use in screening assays. Ideally, one would like to isolate oligomers from patient tissue. However, while the presence of oligomers in vivo has been confirmed (Dimant, H., et al. Acta Neuropathol Commun., 2013. 1:1-10; Hwang, S. S., et al. Analytical Biochemistry, 2019. 566:40-45; Sancesario, G. M., et al. J Alzheimers Dis., 2012. 34:865-878), there are to our knowledge no approaches for isolating amyloid oligomers from patients or of growing and isolating them from animal models of AD. This limitation is compounded by the fact that there is no agreement yet under what conditions and in what cellular compartment these oligomeric species form in vivo. Therefore, development of new oligomer probes requires in vitro amyloid oligomers as substrate. Yet, even generating oligomers in vitro often involves non-physiological solution conditions and it is non-trivial to isolate and stabilize them for subsequent use as assay substrate. Such non-physiological solution conditions required for oligomer stabilization, in turn, can alter the responses of small molecules used in binding assays.

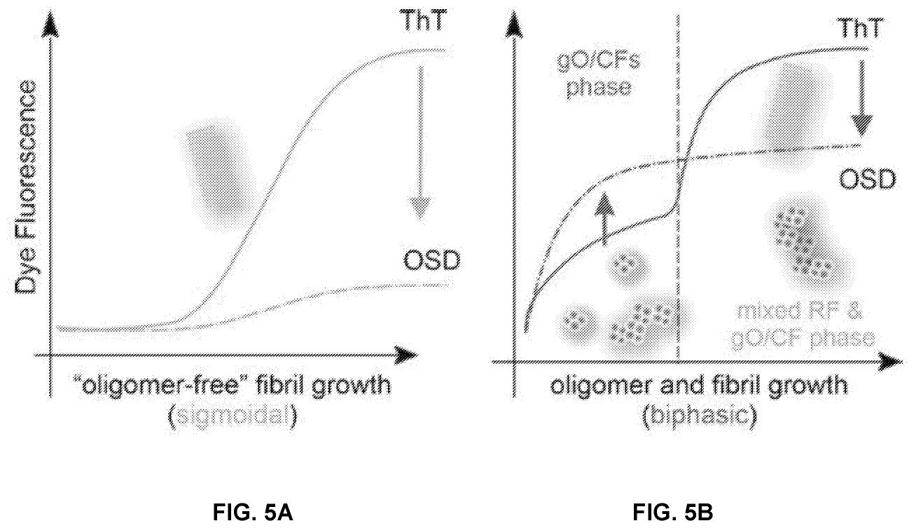

The approach taken for example circumvents the need for isolating oligomers by exploiting a transition in amyloid assembly kinetics from essentially oligomer-free to oligomer dominated fibril growth conditions (FIG. 5). Under suitably chosen conditions, this transition provides well-defined time windows for screening and evaluating the response of small fluorescence dyes to Alzheimer-related oligomers vs. fibrils. In addition, it has been established that for the AD-related Aβ peptides this transition occurs under physiological solution conditions.

Innovation

Our Kinetic Screening Assay Circumvents the Need to Isolate or Stabilize Metastable Oligomers

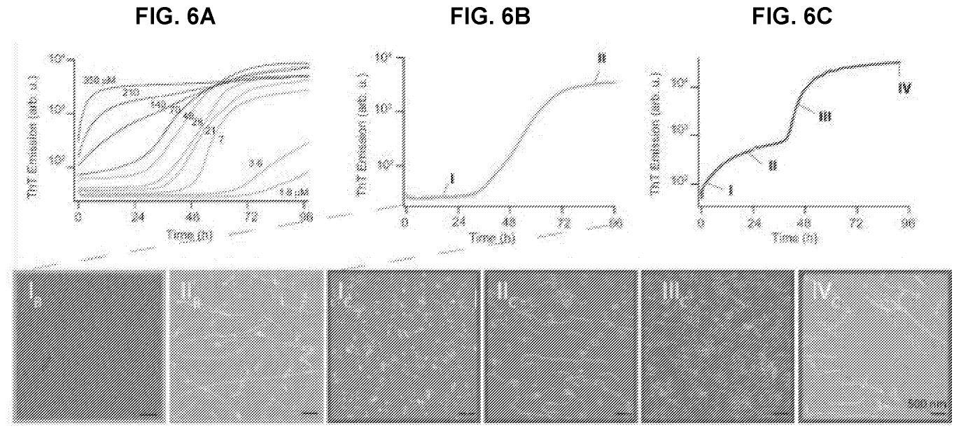

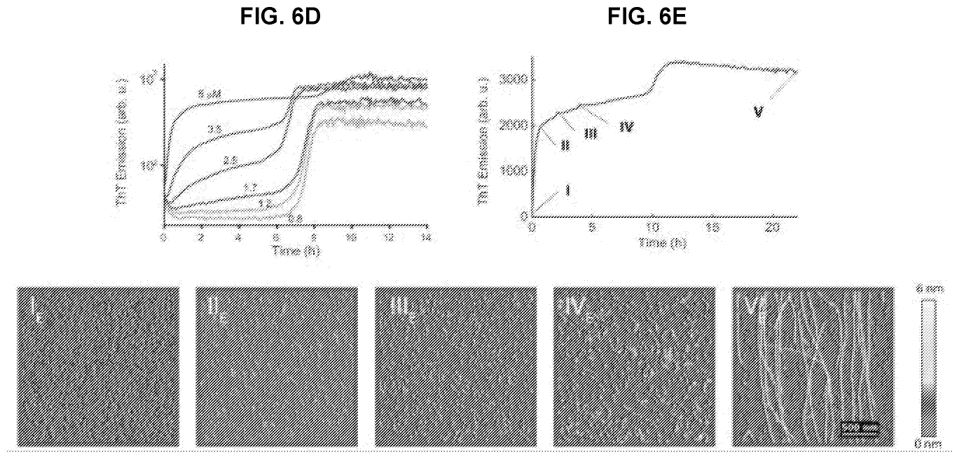

Instead of requiring isolation and stabilization of amyloid oligomers, the dye screening protocol proposed here takes the identification of two distinct amyloid assembly regimes with distinctly different assembly kinetics (see FIG. 6 and Hasecke, F., et al. Chemical Science, 2018. 9:5937-5948). In the first “oligomer-free” regime, fibril assembly displays the standard sigmoidal ThT kinetics associated with nucleated polymerization of fibrils. Following the initial lag phase, this regime yields pure populations of “traditional” rigid fibrils (RFs). It lacks any detectable populations of metastable globular oligomers (gOs) or curvilinear fibrils (CFs) during any stage of fibril assembly. Upon crossing a threshold protein (or salt) concentration a second, oligomer-dominated regime emerges. This regime is characterized by lag-free formation of readily detectable populations of gOs and CFs during its initial phase, with RFs emerging in a second growth phase. The transition to this regime is characterized by a concurrent switch in ThT-monitored assembly kinetics from sigmoidal to biphasic kinetics (Hasecke, F., et al. Chemical Science, 2018. 9:5937-5948; Miti, T., et al. Biomacromolecules, 2015. 16:326-335; Foley, J., et al. J Chem Phys., 2013. 139:121901; Hill, S. E., et al. PLoS ONE, 2011. 6:e18171). Monitoring fluorescence responses of dyes to amyloid assembly under either sigmoidal or biphasic growth conditions provides a direct and straight-forward readout for their selectivity to oligomer over fibril formation.

“Biphasic Oligomers” Match Morphological, Tinctorial and Spectral Characteristics of Toxic Oligomers

As disclosed herein, oligomers and curvilinear fibrils generated during biphasic growth match the morphological, structural and tinctorial characteristics of toxic oligomers and curvilinear fibrils for multiple amyloid diseases, including Alzheimer's disease.

The Disclosed Assay Already Identified an Oligomer-Selective Dye for Lysozyme Amyloid Oligomers

To confirm the potential of the proposed screening approach, it was applied to a small subset of about one dozen fluorescent dyes using the model amyloid hen egg-white lysozyme. This identified crystal violet as oligomer-selective dye under these conditions. While highly promising, we need to identify dyes that recognize Ab oligomers, and do so under near-physiological conditions, which makes them immediately relevant to research and clinical use.

Dye Screening Assays Can Be Readily Extended to the AD Peptides Aβ40 and Aβ42

Conditions were identified for both Aβ40 and Aβ42 that result in the same kinetic transition from sigmoidal to biphasic growth under near-physiological solution conditions. The use of physiological condition for dye screening is important since dye responses tend to be highly sensitive to solution conditions.

OSDs Offer Many Short and Intermediate-Term Advantages Over Existing Modalities of Oligomer Detection

Identification of OSDs would provide an important addition to the limited range of modalities for oligomer detection, and for elucidating the biophysical mechanisms underlying oligomer formation in vivo. The utility of fluorescent dyes is highlighted by the commonly used amyloid fibril indicator thioflavin T (ThT). It offers superior temporal resolution and sensitivity and has been fundamental for developing molecular models of amyloid fibril assembly (Meisl, G., et al. Nature Protocols, 2016. 11:252-272; Knowles, T. P. J., et al. Proc Natl Acad Sci USA, 2011. 108:14746-14751; LeVine, H., et al. Amyloid, 1995. 2:1-6). Its uncharged variant thioflavin S is a frequently used histological amyloid stain. ThT has also served as template for the amyloid binding moiety of the first positron emission tomography (PET) fibril imaging probe (Pittsburgh compound B) in vivo (Klunk, W. E., et al. Annals of Neurology, 2004. 55:306-319). While the weak response of ThT to oligomers was exploited for the assay, its fluorescence response and binding affinity are highly skewed towards late-stage amyloid fibrils (Foley, J., et al. J Chem Phys., 2013. 139:121901). OSDs, in contrast, provides a sensitive fluorescence read-out for detecting and monitoring amyloid oligomer populations in vitro and in vivo (Wu, C., et al. Bioorg Med Chem., 2007. 15:2789-2796). OSDs also provide a favorable starting point for the design of antemortem oligomer detection assays. Similarly, OSDs can be immediately applied for high through-put screening of drugs targeting amyloid oligomers. Perhaps most importantly, just as the development of existing PET tracers utilized the structure of fibril-selective ThT (Klunk, W. E., et al. Annals of Neurology, 2004. 55:306-319), the identification of OSDs represent a highly promising starting point for developing oligomer-selective PET tracers for in vivo imaging. These are all critical steps towards early detection of AD oligomers and, by extension, of early stages of AD in patients. It will also provide essential tools for quantifying the effects of pharmacologic interventions in drug trials targeting Aβ oligomer populations.

Approach

The overall goal of the proposed research is to utilize the rational and efficient kinetics screening assay we developed to identify fluorescent dyes with highly selective responses to Aβ oligomers over fibrils from a library of existing fluorescent dyes. This approach was not only validated but generated data indicating that there are likely multiple already existing dyes that have selective for amyloid oligomers over fibrils.

Background

The Amyloid Cascade Hypothesis and the Role of Amyloid Oligomers

While there is no universal consensus about what causes the clinical symptoms of Alzheimer's Disease (AD), the predominant “amyloid cascade hypothesis” holds that aggregation of the Alzheimer peptides Aβ40 and Aβ42 into fibrils initiates a cascade of downstream events resulting in the clinical symptoms of AD (Hardy, J. A., et al. Science, 1992. 256:184-185). The original fibril-centered hypothesis has since been revised significantly. The cascade hypothesis now accounts for the substantial evidence that amyloid oligomers, small precursors of the prominent late-stage fibril plaques, are critical contributors to and early biomarkers of the etiology of AD (Dahlgren, K. N., et al. J Biol Chem., 2002. 277:36046-36053; Selkoe, D. J., et al. EMBO Mol Med. 2, 2016. 8:595-608; Bitan, G., et al. Amyloid, 2005. 12:88-95; Kayed, R., et al. J Biol Chem., 2004. 279:46363-46366; Kayed, R., et al. Science, 2003. 300:486-489). Hence, understanding the mechanisms underlying oligomer formation in AD and developing assays for the detection of these toxic biomarkers of AD are critical to translate this insight from basic research into practical assays for the diagnosis of early stages of AD, for monitoring disease progression and, subsequently, for evaluating the success of behavioral or pharmacological interventions to halt the progression of this devastating disease.

Progress towards this goal is impeded by the limited ability to confirm where and under what conditions amyloid oligomers form in vivo. In fact, the critical relevance of amyloid oligomers to AD only emerged from in vitro experiments that detected these metastable early-stage precursors of fibrils using biophysical and biochemical techniques. The presence of oligomers in patients' brains was then confirmed using anti-oligomer antibodies—raised against oligomers generated in vitro. Until now, anti-oligomer antibodies remain the predominant modality for attempts at detecting amyloid oligomers ex vivo or in body fluids, and for assessing their spatial distribution throughout the brain (Tomic, J. L., et al. Neurobiology of Disease, 2009. 35:352-358; Savage, M. J., et al. The Journal of Neuroscience, 2014. 34:2884-2897; Koss, D. J., et al. Acta Neuropathologica, 2016. 132:875-895). Small molecule indicators for amyloid oligomers, instead, would provide a highly flexible platform for developing assays for elucidating the progression of oligomer formation under in vitro condition, for detecting amyloid oligomers in body fluids or animal models and, perhaps most importantly, as binding moiety for the development of novel oligomer-selective PET tracers for in vivo imaging.

The overall goal is to screen a library of small dye molecules for fluorescence enhancements upon binding to globular amyloid oligomers (gOs) and their associated curvilinear fibrils (CFs), instead of late-stage rigid fibrils (RFs). For the kinetic screening assay outlined below we do not need to isolate and stabilize these distinct amyloid aggregates species for subsequent dye binding studies. Instead, they emerge naturally as dominant aggregate species within distinct time windows during the assembly reaction (see FIG. 6). In addition, most protocols for generating and maintaining fibril vs. oligomer populations require distinctly different solution conditions or solvents. These, in turn, can significantly affect dye fluorescence and, thereby, prevent a meaningful comparison of fluorescence responses to different amyloid species. The kinetic assay, instead, is performed under fixed solution conditions (temperature, pH, solution composition) and without solvents. It utilizes the (fibril-dominated) response of thioflavin T as reference for dye specific fluorescence responses to oligomers vs. fibrils.

Findings

Introduced below is the kinetic transition in amyloid assembly upon onset of oligomer formation, observed with a dimeric Aβ40 construct (dimAβ) and hen egg-white lysozyme (hewL). Using this screening approach, the dye crystal violet was identified as an oligomer-selective dye (OSD). Next shows was that both of the Alzheimer peptides Aβ40 and Aβ42 display that same kinetic transition upon oligomer formation, allowing extension of the dye screen to the AD-relevant peptides.

Oligomer Formation Induces a Sharp Transition from Sigmoidal to Biphasic ThT Kinetics

The proposed assay is based on the prior observation of a sharp transition, recorded with thioflavin T fluorescence, from sigmoidal to biphasic assembly kinetics as function of monomer or salt concentration, while keeping all other solution conditions fixed (FIG. 6). This transition was identified for two structurally and functionally completely different proteins (hen egg-white lysozyme (hewL) and a dimeric construct of the Alzheimer peptide Aβ40 (dimAβ), grown under widely different solution conditions (Hasecke, F., et al. Chemical Science, 2018. 9:5937-5948; Miti, T., et al. Biomacromolecules, 2015. 16:326-335). In the sigmoidal assembly regime (orange traces in FIGS. 6A, 6B and 6D), amyloid kinetics display the canonical nucleation-polymerization behavior. After an extended lag period without discernible accumulation of any aggregates (FIG. 6, panel I_{B}), the rapid upswing in ThT correlates with the emergence of rigid fibrils (RFs) which are increasing in number and length (FIG. 6, panels II_{B}). Upon crossing a threshold protein concentration, assembly kinetics becomes progressively more biphasic. The initially flat ThT baseline (notice logarithmic scale) first develops a baseline drift that rapidly increases in amplitude with increasing protein concentration. This initial phase is followed by a second upswing in ThT (FIGS. 6C and 6E). During the initial phase, significant populations of globular oligomers (gOs) and curvilinear fibrils (CFs) accumulate (FIG. 6, panels I_{C}, II_{C}) while rigid fibrils start to emerge only during the second upswing (FIG. 6, panels III_{C}, IV_{C}). However, even during this second phase, significant populations of gO/CFs remain and are only replaced by RFs long after the 4 day incubation period shown here (Hasecke, F., et al. Chemical Science, 2018. 9:5937-5948; Miti, T., et al. Biomacromolecules, 2015. 16:326-335). One surprising feature of biphasic growth is that, with increasing monomer concentrations, the ThT plateaus at the end of the incubation periods either don't increase much or even decrease. This is in direct contrast to the sigmoidal regime. Evidence is provided that this is due to the fact that RFs can only nucleate and grow from monomers generated from the glacial depolymerization of metastable gO/CFs s. This also explains the persistence of gO/CFs in AFM images even in the second apparent ThT plateau.

The Transition from Sigmoidal to Biphasic Kinetics can Identify Oligomer-Selective Dyes

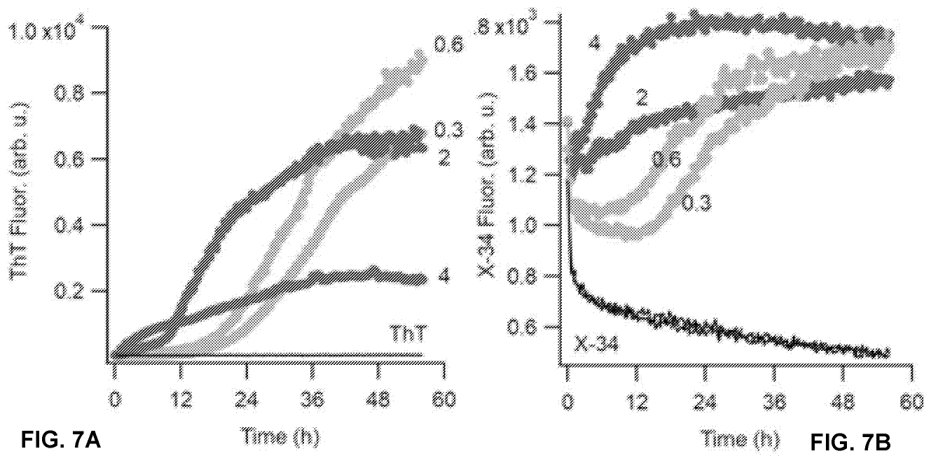

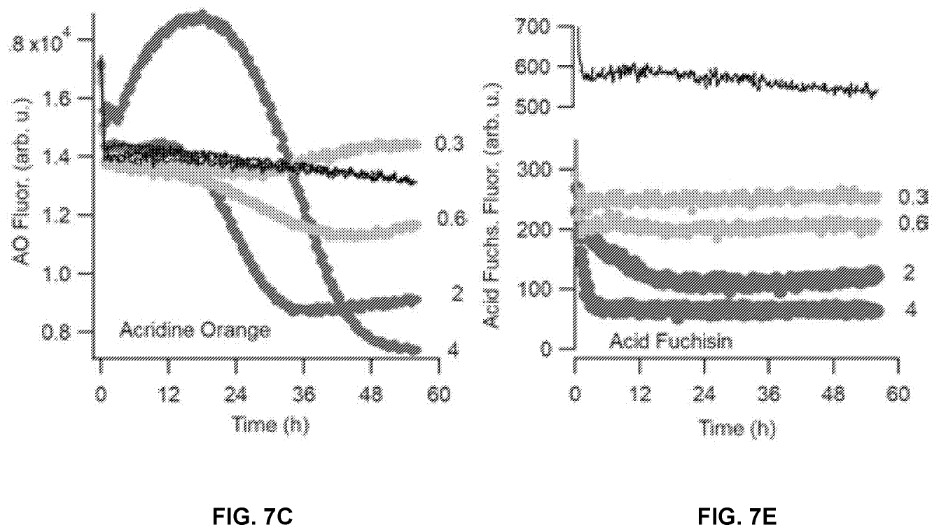

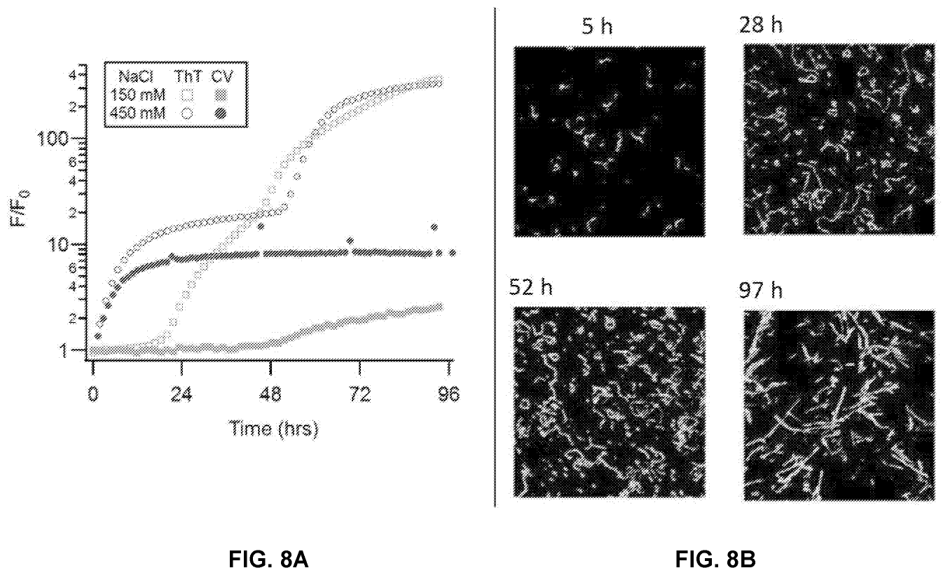

Using the above transition in the model amyloid hewL a small subset of about one dozen dyes were screened for oligomer-selective responses. Oligomer-free sigmoidal kinetics provided the read-out for dye responses to pure fibril formation—relative to the ThT response recorded under the same conditions. Responsiveness of dyes to gO/CF formation was evaluated during the initial gO/CF phase of biphasic kinetics, as long as it was well separated from the second RF-related upswing. Even this very limited and fairly arbitrary set of dyes yielded three different dyes with a modest level of oligomer-specific responses (FIG. 7). While encouraging, the level of oligomer selectivity, the overall fluorescence change upon either fibril or oligomer formation, or the rapid bleaching of the dyes under these growth conditions didn't warrant further investigation. In contrast, the triarylmethane dye crystal violet (and the closely related dye methyl violet) did display noticeable oligomer selectivity (FIG. 8A). In the biphasic regime, both ThT and CV have a robust response to the initial gO/CF dominated phase. In contrast, CV lacks the upswing to subsequent RF nucleation and growth which is readily picked up by ThT. Recording CV and ThT responses during sigmoidal RF growth further confirmed that CV has a weak response to RF formation. FIG. 8B displays the corresponding hewL amyloid aggregate morphologies during bi-phasic growth in the presence of CV. It confirms that the progression of hewL aggregates during biphasic growth is not altered by the presence of CV.

Upon Oligomer Formation, the Alzheimer Peptides Aβ40 and Aβ42 Display Biphasic Kinetics

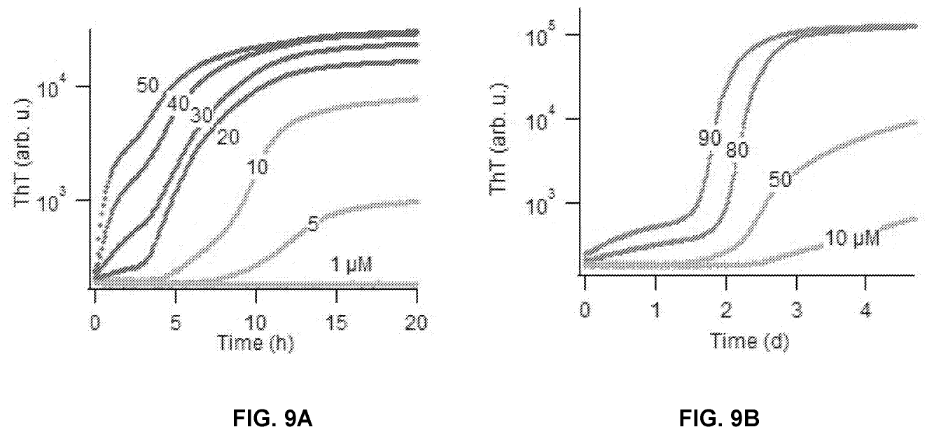

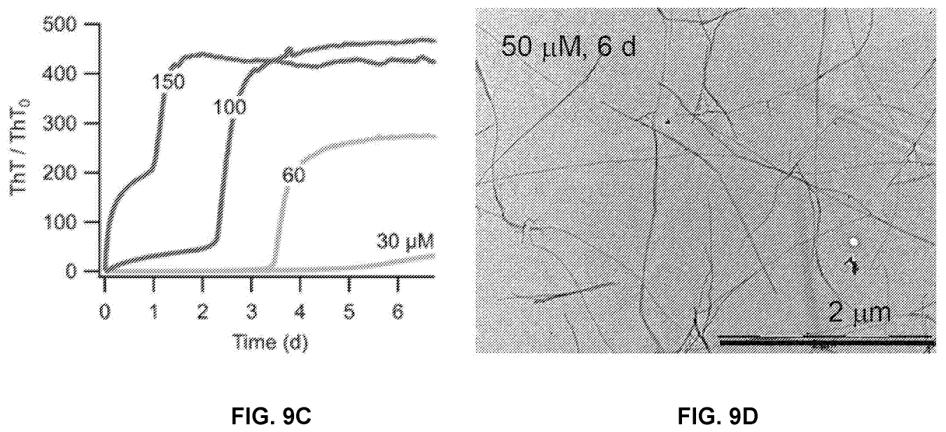

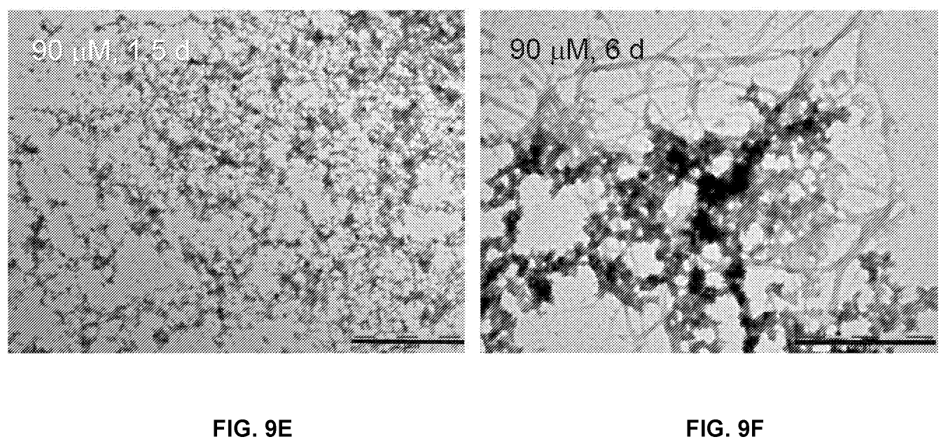

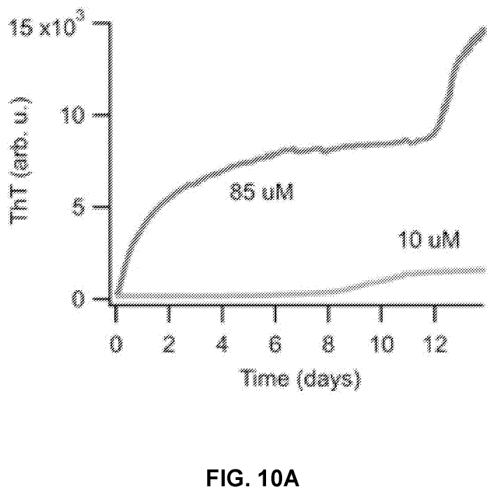

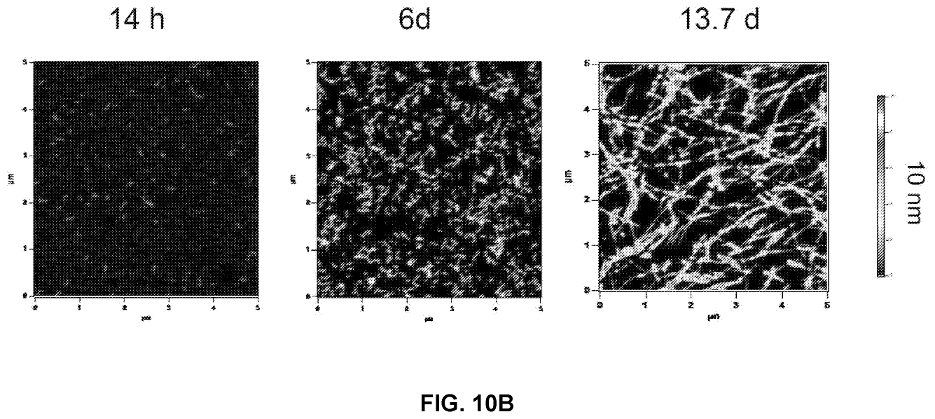

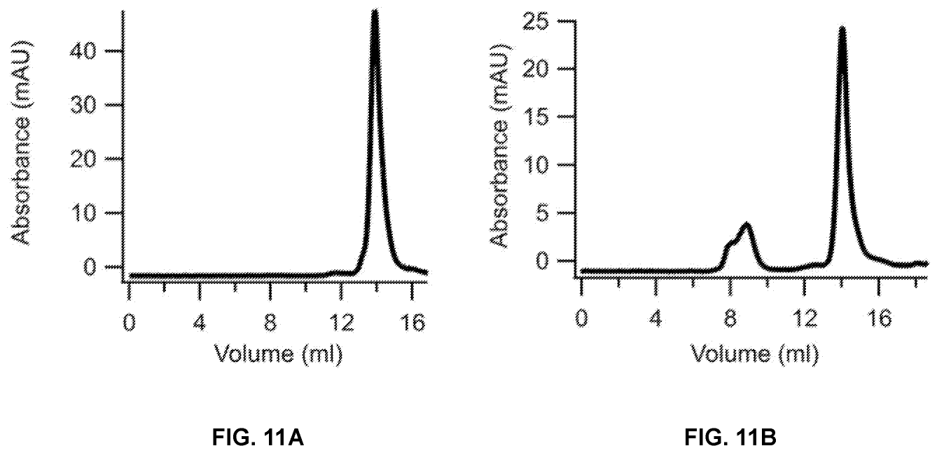

The observations with dimeric Aβ40 strongly suggested that Aβ peptides do have a transition from oligomer-free, sigmoidal to oligomer dominated amyloid growth (see FIG. 6 and Hasecke, F., et al. Chemical Science, 2018. 9:5937-5948). That this is indeed the case is shown in FIG. 9. Both of the AD-related peptides Aβ42 and Aβ40 display the transition from sigmoidal to oligomer-dominated biphasic growth, and do so at physiological pH and near physiological temperatures (27° C.). In addition, our experiments indicate that using physiological NaCl concentrations can further enhanced the oligomer phase of Aβ40 (FIG. 9C). Observations with Aβ are also consistent with prior reports of a threshold concentrations for oligomer formation by Aβ, which should be expected given the pronounced amphiphilic character of Aβ (Sabaté, R., et al. J Phys Chem B., 2005. 109:11027-11032; Soreghan, B., et al. J Biol Chem., 1994. 269:28551-28554). In addition, anti-oligomer antibodies (A11) indicated that, under growth conditions close to ours, Aβ42 oligomers only formed beyond 20 μM (Ladiwala, A. R. A., et al. J Biol Chem., 2012. 287:24765-24773). Similarly, ThT measurements indicated that biphasic kinetics observed with Aβ40 at concentrations near ours were associated with the formation of long-lived amyloid oligomers (Nick, M., et al. Biopolymers, 2018:e23096). Hence, the transition from sigmoidal to biphasic kinetics in Aβ40 and Aβ42 is a reproducible and reliable indicator for oligomer formation by these peptides.

For the dye screening assay it is highly favorable if the initial regime of biphasic kinetics is well separated in time from subsequent fibril nucleation and growth. Hence, Aβ40 under physiological conditions (FIG. 9C) is the focus for the majority of dye screening assays. As additional confirmation that biphasic growth under these conditions replicates our observations of early stage gO/CF vs. late-stage gO/CF and RF populations, ThT kinetics was correlated with their corresponding aggregate morphologies determined with AFM.

Oligomers Formed During Biphasic Growth Share Essential Characteristics of Toxic Oligomers

Aβ42 oligomers grown under biphasic conditions have been shown to react with the anti-oligomer antibody A11 and display the most prominent levels of cellular toxicity (Ladiwala, A. R. A., et al. J Biol Chem., 2012. 287:24765-24773). Similarly, the Prusiner and DeGrado labs have shown that oligomers of Aβ40 derived from biphasic growth are long lived and share structural features associated with toxic species (Nick, M., et al. Biopolymers, 2018:e23096). The morphological and spectroscopic characteristics of biphasic gO/CFs of hewL and dimAβ also match those reported for toxic oligomers in multiple amyloid disease (Hasecke, F., et al. Chemical Science, 2018. 9:5937-5948; Miti, T., et al. Biomacromolecules, 2015. 16:326-335; Foley, J., et al. J Chem Phys., 2013. 139:121901). As determined by AFM, the biphasic gOs display a well-defined globular morphology and progressively polymerize into curvilinear fibrils (CFs) with a characteristic “beads on a string” morphology (AFM panels in FIG. 6). While gOs typically remained unresolved in TEM, CFs display the same “bead on a string” morphology. In contrast to the negative or positive staining patterns for RFs, CFs are stained uniformly by uranyl acetate (FIG. 9E). Thioflavin T fluorescence is considerably less enhanced by gO/CF vs. RF formation (Foley, J., et al. J Chem Phys., 2013. 139:121901). In addition, the CD and ATR-FTIR spectra of biphasic gO/CFs of hewL and dimAβ identify both as amyloid aggregates but with spectral features distinct from those of their fibril counterparts (Hasecke, F., et al. Chemical Science, 2018. 9:5937-5948; Foley, J., et al. J Chem Phys., 2013. 139:121901).

Identify Oligomer-Selective Dyes (OSDs) with Kinetic Screening Assay

Overall Experimental Approach

The central part of the proposed experiments is the determination of dye responses to oligomer vs. fibril growth (i.e. early-stage biphasic vs. late-stage sigmoidal growth), and compare them to those of ThT (see FIGS. 8 & 9). Given the inherent sensitivity of dyes to many environmental factors, it is important that we perform this assay with Aβ peptides under near-physiological growth conditions (see FIGS. 9 & 10).

Preparation of Aβ and Dye Stock for Kinetic Growth Experiments

Lyophilized Aβ40 and Aβ42 stock (e.g. rPeptide, Watkinsville, Ga.) for dye screening assays will be dissolved in 100 mM NaOH (pH 12) and injected into a Superdex 75 10/300 GL column on an FPLC (Äkta Pure, GE) using 35 mM Na₂HPO₄ running buffer at pH 11. The monomer fraction is collected and kept on ice during subsequent sample preparation. Resulting Aβ monomer concentrations will be determined using optical absorption at 280 nm (ε₂₈₀=(1,470±20) M⁻¹cm⁻¹) (Ghosh, P., A. et al. BMC Bioinformatics, 2010. 11:S6-24; Al-Hilaly, Y. K., et al Acta Neuropathol Commun., 2013. 1:83-100). This Aβ stock is then diluted into ice-cold 35 mM Na₂HPO₄ at pH 11 to the highest Aβ concentration used in experiments and the pH is adjusted to pH 7.4 by addition of 1.5% (by vol) of 1M NaH₂PO₄.

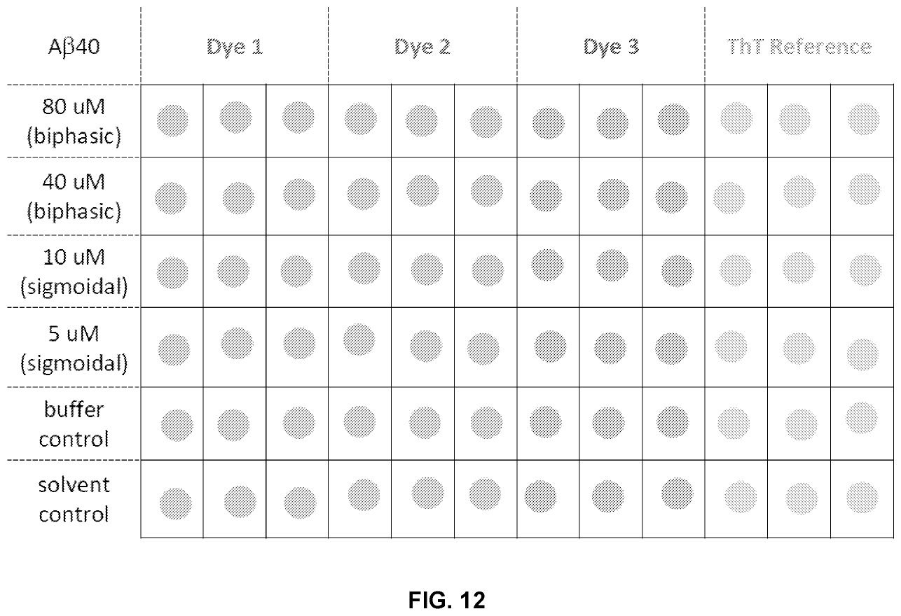

Dye stock solutions are prepared at a typical concentration of 1 mM dye in either distilled water or an appropriate solvent and passed through 220 nm syringe filters. When available, optical absorption coefficients (e.g. ε₄₁₂(ThT)=28,800 M⁻¹ cm⁻¹) will be used to confirm actual dye stock concentrations. Dyes will be diluted into protein solutions at a ratio of approx. 1:100 to a final concentration of 5-10 μM. Dye solvents other than water will be monitored separately for their potential effect on amyloid assembly by running a “dye free” solvent control, with ThT as read-out to confirm unaltered aggregation kinetics (FIG. 12)

Dye Selection Criteria

There are literally tens to hundreds of thousands of dyes one could potentially choose from. Two strategies can be used in dye selection: use already existing amyloid dyes or dyes with potential for oligomer selectivity, or randomly select a subset of dyes from available dye collections. There are multiple dyes that have been suggested to have oligomer selectivity including polarity-sensitive dyes and molecular rotors

Molecular Rotors are widely used amyloid indicator thioflavin T (ThT) belongs to the broad class of molecular rotors with two moieties linked by a rotating bond (Haidekker, M. A., et al., in Chemistry and Biology I: Fundamentals and Molecular Design, A. P. Demchenko, Editor. 2010, Springer Verlag: Berlin. p. 267-308; Haidekker, M. A., et al. J Biol Eng., 2010. 4:11). 9-(Dicyanovinyl) Julolidine (DCVJ) can be included, which has been reported to have some oligomer selectivity (Nagarajan, S., et al. ChemBioChem, 2017. 18:2205-2211). Other molecular rotors to be included are aniline nitriles such as 1,4-dimethylamino benzonitrile (DMABN) and stilbenes such as p-(dimethylamino) stilbazolium (p-DASPMI) and Rhodamine B.

Polarity-Sensitive Dyes can be use. 1-Anilinonaphthalene-8-sulfonic acid (ANS) is frequently used in amyloid aggregation to probe the formation of hydrophobic intermediates (Younan, N. D., et al. Biochemistry, 2015. 54:4297-4306). Therefore, ANS, Bis-ANS, Nile Red and other indicator dyes of hydrophobicity are natural choices in our selection.

Triarylmethane Dyes can be used. Preliminary OSD crystal violet (CV) is a triarylmethane dye. It is best known for staining the peptidoglycan-rich membranes of gram-positive bacteria (Gram, H. C., et al. Fortschritte der Medizin, 1884. 2:185-189). It has previously been identified as a weak metachromatic amyloid stain (Cooper, J. H., J Clin Pathol., 1969. 22:410-413), consistent with the weak fibril response we observe. We will screen at least five additional triarylmethane dyes. This class of dyes is also attractive since several of its members are biocompatible.

Random Selection can be used. Since there are no rational criteria yet to predict dye fluorescence responses to binding or any available oligomer structure to use in binding algorithms, a random subset of dyes from dye catalogs (Molecular Probes) or libraries (e.g. NC States' Max A. Weaver Dye Library) can be used in the dye screen.

Kinetic Dye Screening Assay

In a typical experiment, Aβ40 solutions are prepare at pH 7.4 using at least two Aβ concentrations below (e.g. 5 & 10 μM) and two concentrations above (e.g. 30 & 80 μM) the onset of oligomer formation. A 96 well half-area fluorescence plate (Corning, #3881) are filled with triplicates of each of the four Aβ40 concentrations for each of the dyes used in this screen. This allows for measurements of three different dyes, together with the corresponding ThT reference per assay. The remaining wells are used for controls of dye bleaching in buffer and dye solvent effects, for a total of 72 wells/assay (see FIG. 12 for plate layout). Assay plates will be sealed and incubated slightly above room temperature inside the sample chamber of a Fluostar Omega plate reader (BMG Labtech). ThT fluorescence will be excited with a 445 nm bandpass filter and emission collected using a 482 nm bandpass filter. Appropriate excitation/emission filters will be selected for the dyes used in a given experiment. Typically, fluorescence of all wells will be acquired every 15 min, preceded by a short 10 sec period of gentle plate agitation. The experiment is complete once the sigmoidal solutions have reached their ThT plateau phase.

Data Analysis

The fluorescence dye traces are exported and analyzed using standard data analysis and plotting software (Igor, Wavemetrics). Dye responses will be averaged over their three identical replicas and dye selectivity will be evaluated by normalizing dye responses during biphasic gO/CF vs sigmoidal RFs growth to their fluorescence intensity at t=0 and plotting the ratio F/FO (see FIG. 8A). This fractional fluorescence change (F/FO) will be compared to (a) its overall bleaching behavior under the same conditions (b) any potential solvent effects on dye behavior and (c) the corresponding ThT response under the same conditions.

Anticipated Outcomes and Potential Problems

The screening approach has already been validated with hewL and, as a result, identified crystal violet as OSD (see FIG. 8). In addition, a surprisingly large number of dyes showed (very modest) levels of distinct responses to gO/CF vs. RF formation. In most cases, we anticipate that dyes will remain unresponsive to either type of amyloid growth or that their fluorescence enhancement (fluorescence background FO vs. aggregation-induced enhancement) will be too weak (2-4 fold) to warrant further investigation (see e.g. FIG. 7, panels B-C). Given the initial success rate, it was conservatively anticipate that a total of 5-10 dyes out of the 300 we investigate will show significant potential as OSD. Any of these dyes are scrutinized further (see aim 3).

Correlate OSD Responses to Aβ Oligomers with Immunoreactivity to Anti-Oligomer Antibodies

Tesults from the Tessier and Smith labs indicate that Aβ42 gO/CFs formed under biphasic growth conditions react with All anti-oligomer antibodies (Ladiwala, A. R. A., et al. J Biol Chem., 2012. 287:24765-24773). In addition, the morphological and spectroscopic features of “biphasic gO/CFs” closely match those of toxic oligomers. However, anti-oligomer antibodies are currently the only widely available and accepted read-out for detection of Aβ oligomers in vivo. Therefore, confirming the correlation between dye and antibody responses to “biphasic gO/CFs” strengthens the evidence that the gO/CFs emerging during biphasic growth are identical to those recognized by anti-oligomer antibodies in brain slices and these two approaches for Aβ oligomer detection provide complimentary information.

There are at least two oligomer-selective antibodies commonly used for Aβ oligomer detection: A11 (Kayed, R., et al., in Methods in Enzymology. 2006, Academic Press. p. 326-344; Kayed, R., et al. Science, 2003. 300:486-489) and F11G3 (Lasagna-Reeves, C. A., et al. eLife, 2015. 4:e07558; Guerrero-Muñoz, M. J., et al. Neurobiology of Disease, 2014. 71:14-23; Guerrero-Muñoz, M. J., Biochem Pharmacol., 2014. 88:468-478; Kayed, R, Mol Neurodegener., 2010. 5:57). The immuno-reactivity of both Aβ40 & Aβ42 gO/CF populations generated during biphasic growth to these two anti-oligomer antibodies is determine.

Generating and Isolating “Biphasic gO/CFs” of Aβ40 and Aβ42 Peptide

The dot-blot analysis with anti-oligomer antibodies does require generating and isolating biphasic gO/CF populations. To do so the procedure is followed for analysis of aggregate morphologies and structures with AFM and TEM (see FIG. 6 and 8). Aβ40 or Aβ42 solutions are incubated under biphasic growth conditions (approximately 80 μM for Aβ40 and 30-40 μM for Aβ42, see FIG. 9), the solutions plated into 96-well assay plates and the progress of the assembly reaction monitored with ThT (for details of sample prep and measurements). Aliquots for dot-blot analysis are withdrawn during at least three different time points of the initial phase of biphasic growth. Besides immunoreactivity of each of these aliquots, the morphologies of the aggregates is determine using AFM. Together with ThT kinetics, imaging aggregate morphology provides additional confirmation that the dot-blot analysis is focused on the gO/CF populations generated during the initial phase of biphasic growth.

Dot-Blot Analysis of Anti-Oligomer Reactivity

For the dot-blot analysis the two commercially available anti-oligomer antibodies A11 and F11G3 (Kayed, R., et al., in Methods in Enzymology. 2006, Academic Press. p. 326-344; Kayed, R., et al. Science, 2003. 300:486-489; Guerrero-Muñoz, M. J., Biochem Pharmacol., 2014. 88:468-478; Lasagna-Reeves, C. A., et al. Biochemistry, 2010. 49:10039-10041) are used. Pairs of 5 μL aliquots of Aβ42 or Aβq40 solutions, incubated for different durations in the biphasic growth regime, are blotted onto nitrocellulose membranes, dried and blocked with 10% nonfat milk in TBS, 0.01% Tween 20 for 1 hour. After washing membranes (3 times for 5 minutes) with TBS/0.01% Tween 20, the pairs of blots are exposed for 2 hours to A11 or F11G3 (1 μg/ml each) anti-oligomer antibodies, respectively. Following a second washing step, membranes are incubated with anti-rabbit IgG conjugated with horseradish peroxidase (1:10,000) at room temperature for 1 h. Antibody staining is quantified using the Super signal West pico chemiluminescence kit from Thermo Scientific and imaged on a Biorad ChemDoc-Touch Imaging System.

Anticipated Outcomes and Potential Problems

Since previous experiments already indicated the reactivity of A11 with biphasic gO/CFs (Ladiwala, A. R. A., et al. J Biol Chem., 2012. 287:24765-24773), this result is expected. However, there could be variability in the reactivity that these antibodies show to early-stage gOs versus their polymerized CF counterparts. Correlated AFM images permit evaluation of whether these antibodies recognized both types of gO-related populations or whether part of the anti-oligomer epitope becomes inaccessible upon polymerization into CFs. This, in and of itself would be an interesting finding. In addition, it is possible that A11 and F11G3 display distinct reactivity to Aβ40 vs. Aβ42 gO/CFs. If so, such select responses to oligomeric subspecies might be reflected in the fluorescence responses of a given OSD, as well. Structure-specific dye responses have been observed recently for Aβ fibrils (Condello, C., et al. Proc Natl Acad Sci USA, 2018. 115:E782-E791). Such variability would make OSDs an even more versatile tool for detecting and characterizing Aβ oligomers.

Validation and Characterization of OSDs

Dyes identified as potential OSDs by the kinetic assay are subjected to additional scrutiny and characterization. Specifically, “false positives” i.e. dyes that selectively inhibit RF growth and, thereby, mimic preferential affinity for gO/CFs, are excluded. Fluorescence spectra of OSDs bound to gO/CFs are determine. Any spectral shifts upon binding would further improve specificity of OSD responses to oligomers over unrelated environmental factors altering their fluorescence intensity only. Whether any given OSD can be used with ThT to separate in vitro Aβ amyloid growth into its underlying gO/CF and RF components is evaluated. This would provide a new screening assay for monitoring the separate effects of drugs on oligomer vs. fibril populations.

Excluding Fibril Inhibitors as False Positives

The kinetic assay might identify “false positives”, i.e. dyes for which the apparent oligomer-selectivity arises from dye-mediated inhibition of RF kinetics, instead (Necula, M., et al. J Biol Chem., 2007. 282:10311-10324). Since the goal is to find dyes with select staining of existing oligomers over fibrils, dyes are excluded which are fibril growth inhibitor, instead. Two approaches are used to test during with lysozyme and crystal violet: competitive inhibition and direct AFM imaging. For competitive inhibition a solution under sigmoidal growth conditions is incubate with ThT only and with ThT and series of increasing concentration of added OSD. Any fibril inhibitor can alter not only the amplitudes but also slow the time course of ThT responses (lag periods, rise time etc). To discriminate changes in the time-dependent ThT fluorescence from reduced ThT amplitudes due to competitive binding of the OSD to fibrils, the fractional ThT responses are determined for each OSD concentration

Δ(ThT)=(ThT(t)−ThT[0])/(ThT(max)−ThT[0])

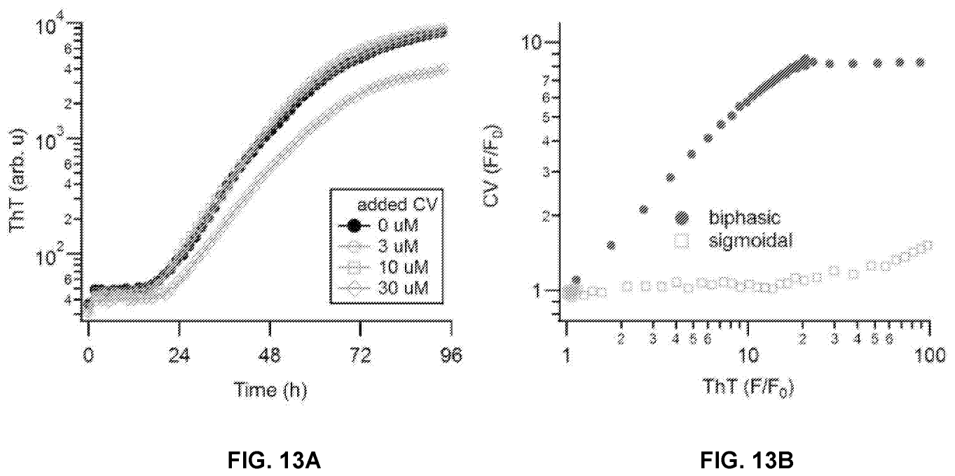

and plotting those curves on top of each other. A tight superposition suggests that the given OSD does not alter RF kinetics significantly—at least up to some critical value (see FIG. 13A). Furthermore, the kinetics of the ThT and OSD signal for identical solutions undergoing sigmoidal fibril growth should have a one-to-one temporal correlation. This has been established for CV and ThT during sigmoidal and the initial phase of biphasic growth of hewL amyloid RFs (see FIG. 13B). As second confirmation, the endpoints of fibril assembly is imaged in the presence or absence of added OSD concentration using TEM imaging. The typical density of RFs under these conditions will also identify any noticeably inhibition of RF nucleation and growth by an OSD.

Fluorescence Spectra of OSDs Upon Binding to gO/CFs

the excitation and emission spectra for potential OSDs bound to Aβ40 gO/CFs is determined. As known for ThT, fluorescence spectra in the amyloid-bound state can shift noticeably relative to their unbound state (Singh, P. K., et al. Chemical Communications, 2015. 51:14042-14045). To determine OSD spectra in their bound state, Aβ40 is incubated under biphasic growth conditions that result in an extended gO/CFs plateau. However, solutions are placed into a sealed small-volume fluorescence cuvette and incubated in the temperature-controlled sample chamber of a spectrofluorometer (Fluoromax-4, Horiba). OSD fluorescence spectra is acquired using the unbound excitation/emission values. Once the initial plateau phase is reached, though, the kinetic measurement are stopped and the relevant excitation/emission spectra in the presence of gO/CFs determined using the following iterative process. Using the initial excitation wavelength, an emission spectrum is acquired over a wide range of wavelengths. Next an excitation spectrum is acquired using the peak wavelength identified in the previous emission scan as detection wavelength. This procedure is repeated until no further optimization of the peak excitation/emission values is observed. Comparing the initial dye spectra to those obtained during the gO/CF phase will indicate whether the given OSD does undergo a spectral shift.

Separating gO/CF from RF Kinetics Using Simultaneous OSD and ThT Recordings

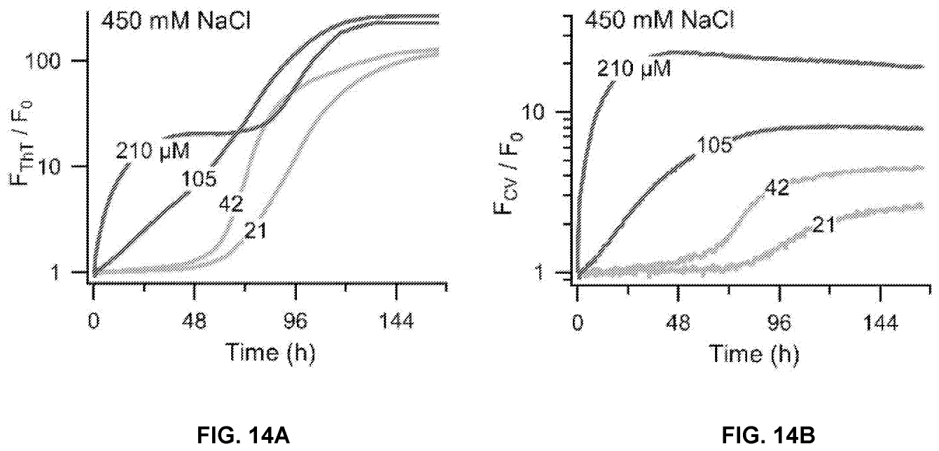

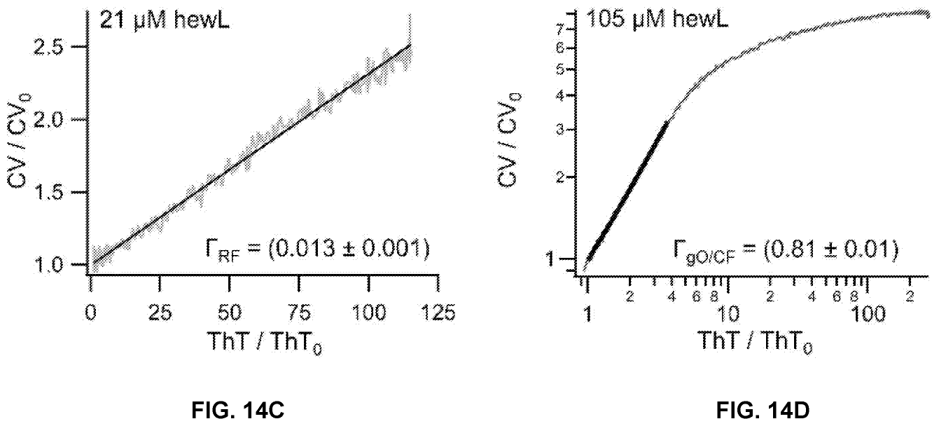

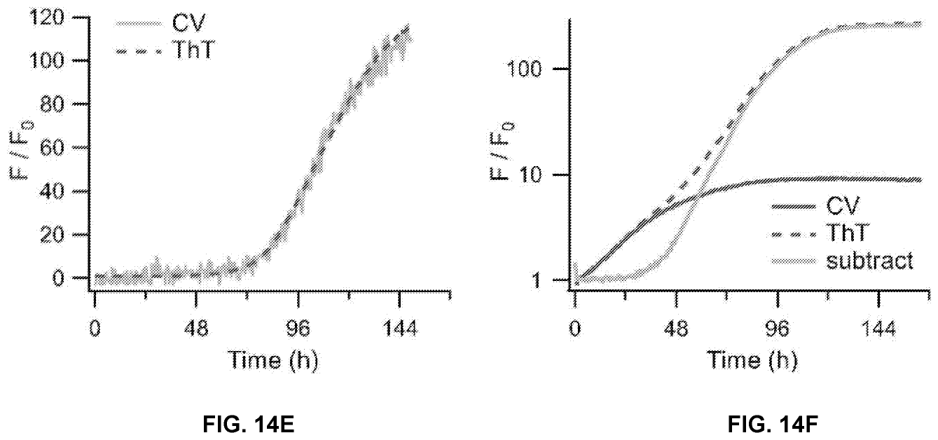

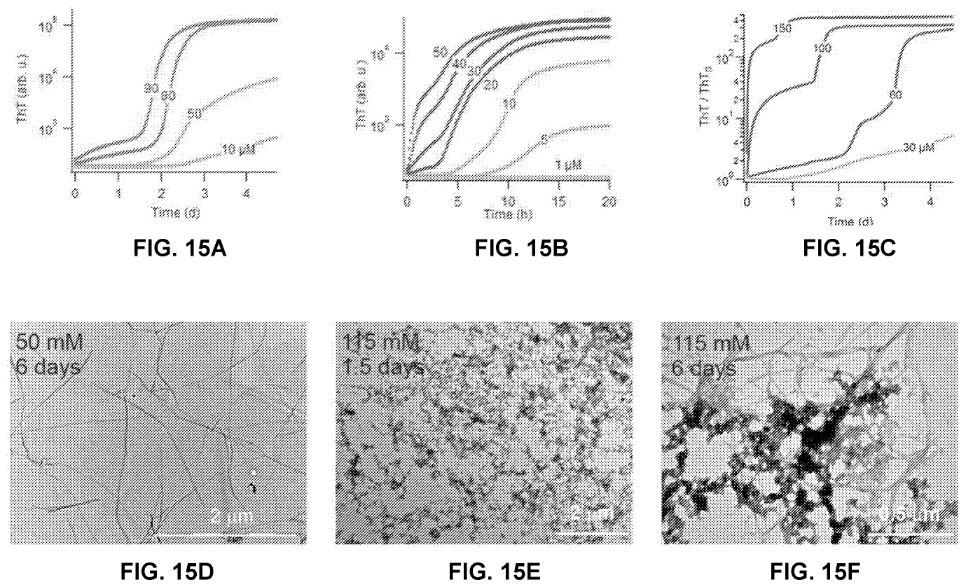

One immediate application of OSDs is their use in high throughput screening of drug compounds targeting Aβ oligomer formation. However, it would be significantly more useful to monitor the effect of a given drug on both gO/CF and RF populations simultaneous, and in the process determine how an effect on e.g. oligomer formation affects fibril formation or vice versa. Simultaneous measurements of OSD and ThT fluorescence could provide such an assay. There are several criteria an OSD has to meet for simultaneous recordings, though. First of all, OSD excitation or emission (or both) wavelengths need to be sufficiently different from ThT to avoid an overlap in their responses. Similarly potential effects of a given OSD on the assembly process itself or its associated ThT signal should be excluded. The above two types of dye characterization will have addressed those concerns already. Here determination is made whether simultaneous recordings of OSD and ThT signals from Aβ40 solutions undergoing biphasic growth permit separating these two fluorescence signals into their underlying gO/CF and RF components. The same protocol we established for CV and ThT during sigmoidal RF vs. biphasic gO/CF and RF formation (see FIG. 14) is followed. Triplicates of Aβ40 solutions are incubated at identical growth conditions but at two Aβ40 concentrations resulting in sigmoidal RF growth and two concentrations leading to biphasic gO/CF and RF growth. The OSD under investigation and 10 μM of ThT are added to each of these wells and the fluorescence recorded until all four solutions have reached the ThT plateau of RF growth (FIGS. 14A, 14B). Fractional ThT and OSD fluorescence at identical measurement times are plotted against each other (see FIGS. 14C, 14D). In the initial phase of the biphasic regime, this provides the correlation between OSD and ThT responses to gO/CF formation. By multiplying either the fractional change in OSD or ThT fluorescence with this correlation factor, the responses of the two dyes to gO/CF formation can be matched to each other. Subtracting the matched OSD from the ThT trace should therefore remove the contributions of gO/CF formation to the ThT signal and recover the purely sigmoidal curve for RF formation.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A system, comprising
a rigid fibril (RF)-selective dye, and
a globular amyloid oligomers (gOs) and curvilinear fibrils (CFs)-selective dye.

2. The system of claim 1, wherein the RF-selective dye comprises thioflavin T.

3. The system of claim 1, wherein the gO/CF-selective dye comprises a methyl violet dye.

4. The system of claim 3, wherein the gO/CF-selective dye comprises triarylmethane crystal violet.

5. The system of claim 1, wherein the RF-selective dye and gO/CF-selective dye are present in a composition.

6. The system of claim 1, wherein the RF-selective dye and gO/CF-selective dye are present in a kit.

7-8. (canceled)

9. An in vitro method for detecting amyloids in a sample or tissue, comprising
(a) mixing the sample or contacting the tissue with a globular amyloid oligomers (gOs) and curvilinear fibrils (CFs)-selective dye; and
(b) assaying the sample or tissue either for the time-evolution or total amount of gO/CF present in the solution.

10. The method of claim 9, wherein the gO/CF-selective dye comprises a methyl violet dye.

11. The method of claim 10, wherein the gO/CF-selective dye comprises triarylmethane crystal violet.

12. The method of any one claim 9, further comprising
(c) mixing the sample or contacting the tissue with a rigid fibril (RF)-selective dye; and
(d) assaying the sample or tissue either for the time-evolution or total amount of RF-present in the solution.

13. The method of claim 12, wherein the RF-selective dye comprises thioflavin T.

14-18. (canceled)

19. An in vivo method for detecting amyloids in a subject, comprising
(a) administering to the subject a composition comprising a globular amyloid oligomers (gOs) and curvilinear fibrils (CFs)-selective dye; and
(b) assaying the subject for presence of the gO/CF-selective dye.

20. The method of claim 19, wherein the gO/CF-selective dye comprises a methyl violet dye.

21. The method of claim 20, wherein the gO/CF-selective dye comprises triarylmethane crystal violate.

22. The method of claim 19, further comprising
(c) administering to the subject a composition comprising rigid fibril (RF)-selective dye; and
(d) assaying the subject for presence of the RF-binding dye.

23. The method of claim 22, wherein the RF-selective dye comprises thioflavin T.
